(19) United States
(12) Patent Application Publication
(10) Pub. No.: US 2021/0020325 A1
(43) Pub. Date: Jan. 21, 2021

(54) BOWTIE FILTER, RADIATION SCANNING APPARATUS, AND RADIATION SCANNING METHOD

(71) Applicant: OUR UNITED CORPORATION, Xi'an (CN)

(72) Inventors: Hao Yan, Xi'an (CN); Zhao Guo, Xi'an (CN); Jinsheng Li, Xi'an (CN)

(21) Appl. No.: 17/043,260

(22) PCT Filed: Nov. 13, 2018
(86) PCT No.: PCT/CN2018/115269

§ 371 Date: Sep. 29, 2020

(51) Int. Cl. G21K 1/10; A61N 5/10

(52) U.S. Cl. CPC G21K 1/10; A61N 2005/1095; A61N 5/1043

(57) ABSTRACT

The present disclosure discloses a bowtie filter, a radiation scanning apparatus, and a radiation scanning method, and is used for improving the versatility of the bowtie filter, simplifying the operation of radiation scanning, and improving the efficiency of radiation scanning. The bowtie filter includes a filter body having at least two filter regions. Each of the at least two filter regions is in contact with or partially coincident with an adjacent filter region, and every two adjacent filter regions have different radiation compensation amounts. The radiation scanning apparatus includes a radiation source and the bowtie filter. The bowtie filter is disposed at a light outlet side of the radiation source, and each filter region of the bowtie filter corresponds to a different irradiation field of the radiation source.

### CROSS-REFERENCE TO RELATED APPLICATION

This application is a national phase entry under 35 USC 371 of International Patent Application No. PCT/CN2018/115269 filed on 13 Nov. 2018, which is incorporated herein by reference in its entirety.

### TECHNICAL FIELD

The present disclosure relates to the technical field of medical equipment, and in particular, to a bowtie filter, a radiation scanning apparatus, and a radiation scanning method.

### BACKGROUND

During tumor scanning on a patient's head or body, in order to reduce a scanning radiation dose and reduce effects of beam hardening and scattering, and improve quality of a scanned image, a bowtie filter is generally disposed between an X-ray source and the patient. Since for different patients, body parts, tumor locations and tumor sizes that need to be scanned are different, different types of bowtie filters are generally needed to be selected to suit the patients.

Currently, one of the most common methods is to manually replace the bowtie filters to ensure that the bowtie filters applied to different patients are capable of meeting the needs of corresponding patients. Of course, in order to improve an efficiency of replacing the bowtie filters, a filter adjustment component may also be used to automatically switch different bowtie filters. For example, different types of bowtie filters are respectively disposed on different sliders of the filter adjustment component. By controlling a movement of each slider on a corresponding guide rail, a bowtie filter suitable for the patient may be kept at a filtering position. However, whether the bowtie filters are manually replaced or automatically switched, different types of bowtie filters are continuously disassembled and assembled or switched according to different scanning needs of the patients, which is cumbersome to operate, and easy to increase a difficulty of radiation scanning and consume a large labor cost. Therefore, it is not conducive to an efficient execution of the radiation scanning.

### SUMMARY

An objective of the present disclosure is to provide a bowtie filter, a radiation scanning apparatus, and a radiation scanning method, which are used to improve the versatility of the bowtie filter, simplify an operation of the radiation scanning, and improve an operation efficiency of the radiation scanning.

In order to achieve the above objective, the present disclosure provides the following technical solutions.

In a first aspect, the present disclosure provides a bowtie filter, and the bowtie filter includes a filter body, and the filter body includes at least two filter regions. Each of the at least two filter regions is in contact with or partially coincident with an adjacent filter region, and every two adjacent filter regions have different radiation compensation amounts.

In the bowtie filter provided by the present disclosure, at least two filter regions are disposed on a same filter body, and each of the at least two filter regions is in contact with or partially coincident with an adjacent filter region, so that every two adjacent filter regions have different radiation compensation amounts, which may effectively improve the versatility of the bowtie filter, that is, at least two different filtering effects may be achieved with a same bowtie filter, so as to meet at least two different scanning requirements of the users, thereby appropriately reducing a replacement frequency of the bowtie filter. Moreover, a structure of the bowtie filter provided by the present disclosure is simple, and the bowtie filter provided by the present disclosure may be used to perform different degrees of radiation compensation by adjusting an exit angle of a corresponding radiation source, which greatly simplifies an operation of radiation scanning corresponding to different scanning requirements, and is advantageous for reducing an operation difficulty of the radiation scanning, and reducing consumption of labor costs, thereby improving the operation efficiency of the radiation scanning.

Based on the above bowtie filter, in a second aspect, the present disclosure provides a radiation scanning apparatus, and the radiation scanning apparatus includes a radiation source and a bowtie filter. The bowtie filter includes a filter body, and the filter body includes at least two filter regions. Each of the at least two filter regions is in contact with or partially coincident with an adjacent filter region, and every two adjacent filter regions have different radiation compensation amounts. The bowtie filter is disposed at a light outlet side of the radiation source, and each filter region of the bowtie filter corresponds to a different irradiation field of the radiation source.

Based on the radiation scanning apparatus, in a third aspect, the present disclosure provides a radiation scanning method, and the method includes: determining a filter region from filter regions of the bowtie filter as a target filter region according to a scanning requirement of a user; controlling radioactive rays emitted from a radiation source to pass through the target filter region to irradiate a portion to be scanned of the user, an irradiation field of the radiation source corresponding to the target filter region being a target irradiation field; and redetermining another filter region as the target filter region from the filter regions of the bowtie filter in a case where a scanning requirement of a user is changed, and correspondingly switching another irradiation field of the radiation source as the target irradiation field.

The beneficial effects that may be achieved by the radiation scanning apparatus and the radiation scanning method provided by the present disclosure are the same as the beneficial effects of the bowtie filter provided by the above technical solution, which will not be described here again.

### BRIEF DESCRIPTION OF THE DRAWINGS

FIG. 1 is a schematic diagram showing a structure of a bowtie filter, in accordance with embodiments of the present disclosure;

FIG. 2 is a schematic diagram showing a structure of another bowtie filter, in accordance with embodiments of the present disclosure;

FIG. 3 is a schematic diagram showing a structure of yet another bowtie filter, in accordance with embodiments of the present disclosure;

FIG. 4 is a schematic diagram showing a structure of yet another bowtie filter, in accordance with embodiments of the present disclosure;

FIG. 5 is a schematic diagram showing a structure of yet another bowtie filter, in accordance with embodiments of the present disclosure;

FIG. 6 is a schematic diagram showing a structure of a radiation scanning apparatus, in accordance with embodiments of the present disclosure;

FIG. 7 is a schematic diagram showing ranges of a head irradiation field and a body irradiation field in the radiation scanning apparatus shown in FIG. 6; and

FIG. 8 is a flow diagram of a radiation scanning method, in accordance with embodiments of the present disclosure.

### DETAILED DESCRIPTION

In order to further describe the bowtie filter, the radiation scanning apparatus, and the radiation scanning method provided by the embodiments of the present disclosure, a detailed description will be made with reference to the accompanying drawings.

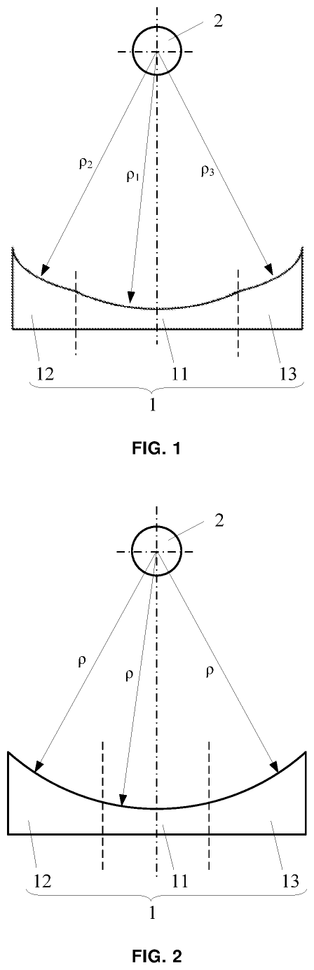

Referring to FIGS. 1 to 3, the bowtie filter provided by the embodiments of the present disclosure includes a filter body 1 having at least two filter regions. Each of the at least two filter regions is in contact with or partially coincident with an adjacent filter region, and every two adjacent filter regions have different radiation compensation amounts.

The number of filter regions in the filter body 1, a structure and a radiation compensation amount of each filter region may be set according to actual needs. In some embodiments, the filter body includes three filter regions as shown in FIGS. 1 and 2, and the three filter regions are a first filter region 11, a second filter region 12 and a third filter region 13. The first filter region 11 is used for radiation compensation of a head to be scanned. The second filter region 12 and the third filter region 13 are both used for radiation compensation of a body to be scanned. The second filter region 12 and the third filter region 13 may have the same or different radiation compensation amounts. Of course, the third filter region 13 may also be used for radiation compensation of other scanning requirements in addition to head scanning and body scanning, and a structure and a corresponding radiation compensation amount of the third filter region 13 is specifically set according to specific needs.

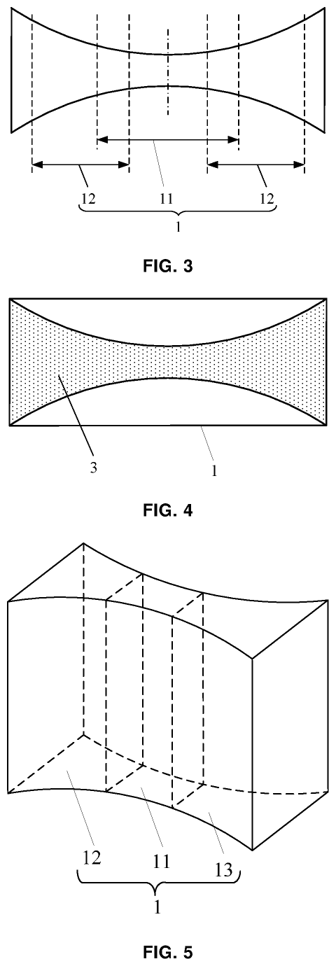

It will be noted that the above filter regions are disposed in a same filter body 1 and are used for performing different degrees of radiation compensation for a same radiation source 2, and the filter regions may be sequentially arranged in the filter body 1 along a circumferential direction of the radiation source 2. In order to achieve a seamless switching between adjacent filter regions and effectively improve space utilization of the filter body, sidelines of each filter region and an adjacent filter region, such as the first filter region 11 and the second filter region 12 shown in FIG. 1, may be coincident and jointed. Or a partial region of each filter region may be coincident with that of an adjacent filter region, such as the first filter region 11 and the second filter region 12 shown in FIG. 3, and in a case where two adjacent filter regions are partially coincident, irradiation fields corresponding to the radiation source are also partially coincident.

In the bowtie filter provided by the embodiments of the present disclosure, at least two filter regions are provided in a same filter body 1, and each of the at least two filter regions is in contact with or partially coincident with an adjacent filter region, so that every two adjacent filter regions have different radiation compensation amounts, which may effectively improve the versatility of the bowtie filter. That is, a same bowtie filter may be used to achieve at least two different filtering effects, so as to meet at least two different scanning requirements of the user, thereby appropriately reducing a replacement frequency of the bowtie filter. Moreover, the structure of the bowtie filter provided by the embodiments of the present disclosure is simple, and the bowtie filter provided by the embodiments of the present disclosure may be used to perform different degrees of radiation compensation by adjusting an exit angle of a corresponding radiation source, which greatly simplifies the operation of radiation scanning corresponding to different scanning requirements, and is advantageous for reducing an operation difficulty of the radiation scanning, and reducing consumption of labor costs, thereby improving the operation efficiency of the radiation scanning.

It will be understood that the radiation scanning that the user needs to perform on a daily basis may be generally divided into two categories: a head scanning and a body scanning. Therefore, in order to ensure high versatility of the bowtie filter, referring to FIGS. 1 to 3, in the bowtie filter provided in the embodiment, the at least two filter regions include at least one first filter region 11 used for radiation compensation of the head to be scanned, and at least one second filter region 12 used for radiation compensation of the body to be scanned. Since the radiation compensation amounts required for the user's head scanning and body scanning are not the same, a surface of the first filter region 11 facing the radiation source 2 may be set to have a shape of the head of the user, and a surface of the second filter region 12 facing the radiation source 2 may be set to have a shape of the body of the user.

Optionally, a surface of the first filter region 11 used for facing the radiation source 2 is a first curved surface, a surface of the second filter region 12 used for facing the radiation source 2 is a second curved surface, and a curvature center of the first curved surface and a curvature center of the second curved surface are in a same straight line or coincident. For example, radioactive rays emitted from the radiation source directly irradiate the filter body 1, and the curvature center of the first curved surface and the curvature center of the second curved surface are in a same straight line or coincident with a light source center of the radiation source. In addition, if the filter body 1 is further provided with a third filter region 13, similarly, a surface of the third filter region 13 used for facing the radiation source 2 is a third curved surface, and a curvature center of the third curved surface and the curvature center of the first curved surface are in a same straight line or coincident.

In some embodiments, referring to FIG. 1, curved surfaces of each filter region used for facing the radiation source 2 in the filter body 1 may have different curvature or curvature variations respectively. For example, curvature of the first curved surface of the first filter region 11 is ρ1, curvature of the second curved surface of the second filter region 12 is ρ2, and curvature of the third curved surface of the third filter region 13 is ρ3, wherein ρ1 is not equal to ρ2, and ρ2 is not equal to ρ3. Further, in these embodiments, in a case where the first curved surface of the first filter region 11 is disposed adjacent to the second curved surface of the second filter region 12, the first curved surface of the first filter region 11 is smoothly in contact with the second curved surface of an adjacent second filter region 12, that is, the first curved surface of the first filter region 11 and the second curved surface of the second filter region 12 are smoothly transitioned. The adjacent curved surfaces of the other different filter regions are disposed in a similar fashion. In this way, sharp protrusions or bad cusps may be avoided forming on a surface of the filter body 1 facing the radiation source 2, which avoids adverse effects on the generation of scanned images of the radiation scanning.

In some other embodiments, referring to FIG. 2, curved surfaces of each filter region used for facing the radiation source 2 in the filter body 1 may have same curvature or curvature variation. For example, the first curved surface of the first filter region 11 and the second curved surface of the second filter region 12 maintain constant curvature p, and the curvature of the two are the same. Different radiation compensation amounts of the first filter region 11 and the second filter region 12 are mainly realized by the first filter region 11 and the second filter region 12 being respectively located in different volumes corresponding to irradiation fields of the radiation source. Of course, in a case where the first curved surface of the first filter region 11 and the second curved surface of the second filter region 12 are variable curvature surfaces, a curvature variation of the first curved surface of the first filter region 11 may be the same as a curvature variation of the second curved surface of the second filter region 12. Based on this, a curved surface of the filter body 1 composed of the first curved surface of the first filter region 11 and the second curved surface of the second filter region 12 is smooth, which is not only convenient for processing, but also capable of acquiring a high-quality radiation scanned image.

In some embodiments, referring to FIG. 3, the filter body 1 of the bowtie filter is provided with three filter regions, which are one first filter region 11 and two second filter regions 12, and the two second filter regions 12 are respectively disposed on both sides of the first filter region 11. The two second filter regions 12 are used for radiation compensation of the body to be scanned, and may have a same radiation compensation amount. The two second filter regions 12 are symmetrically disposed with the first filter region 11 as a center, that is, the second curved surfaces of the two second filter regions 12 have same curvature or curvature variation. The two second filter regions 12 may also be set as structures having different radiation compensation amounts, that is, the second curved surfaces of the two second filter regions 12 have different curvature or curvature variations.

It is worth mentioning that, in the above embodiment, the first filter region 11 is used for radiation compensation of the head to be scanned, and the second filter region 12 is used for radiation compensation of the body to be scanned. In a case where the first curved surface of the first filter region 11 and the second curved surface of the second filter region 12 have the same curvature ρ, curvature of the curved surface required for the radiation compensation of the head to be scanned is a head applicable curvature ρₜ₁, and curvature of the curved surface required for the radiation compensation of the body to be scanned is a body applicable curvature ρₜ₂. Since the head applicable curvature ρₜ₁ is generally greater than the body applicable curvature ρₜ₂, the curvature ρ of the first curved surface and the second curved surface may generally be selected between the head applicable curvature ρₜ₁ and the body applicable curvature ρₜ₂. That is, ρₜ₂ is less than ρ, and ρ is less than ρₜ₁. In addition, in order to reduce a design difficulty of the bowtie filter and facilitate the fabrication, it is also acceptable to adopt the body applicable curvature ρₜ₂ as the above curvature ρ of the first curved surface and the second curved surface.

In order to evenly distribute the filter regions having different radiation compensation amounts, referring to FIGS. 3 and 5, the embodiment provides another bowtie filter. The filter body 1 of the bowtie filter is a columnar filter body. A cross section of the columnar filter body is in a bow-tie shape. For example, a central region of the bow in the filter body 1 is provided with a first filter region 11. The other filter regions are sequentially in contact with both sides of the first filter region 11 along a direction in which butterfly wings of the bow are unfolded, for example, the second filter region 12 and the third filter region 13 shown in FIG. 5.

It will be noted that the bowtie filter in the above embodiment is used for radiation compensation, and the filter body is generally made of material with good radiation attenuation properties. For example, the above filter body 1 includes an aluminum filter body, a ceramic filter body or a teflon filter body, that is, the filter body 1 may be made of aluminum metal, ceramic material or polytetrafluoroethylene (PTFE) material.

In addition, the above filter body 1 may also be composed of a light-transmitting substrate and heavy metal compound particles 3 doped in the light-transmitting substrate. The light-transmitting substrate allows visible light to pass through, and is generally made of transparent or translucent material such as glass or light-transmitting resin and the like. The heavy metal compound generally has high radiation attenuation properties. In the embodiment of the present disclosure, the heavy metal compound particles are doped in the light-transmitting substrate, and the radioactive rays emitted from the radiation source may be compensated for a certain amount by using the heavy metal compound particles having a certain density. Optionally, the above heavy metal compounds include a compound of at least one metal element of lead, chromium, tin, nickel, cobalt, antimony, cadmium, or bismuth. For example, the above filter body adopts a glass filter body doped with heavy metal compound particles. The glass filter body is lead glass, and the heavy metal compound particles included therein are lead oxide particles.

It will be added that in a case where the filter body 1 is composed of the light-transmitting substrate and the heavy metal compound particles doped in the light-transmitting substrate, each filter region may be formed by shaping the heavy metal compound particles in a corresponding region in the light-transmitting substrate. That is to say, a shape of each filter region is related to a distribution shape of the heavy metal compound particles. Optionally, the glass filter body is formed by fitting the shapes of the filter regions, such as the bow column shown in FIG. 5, and the heavy metal compound particles are uniformly distributed in the glass filter body. Or, the glass filter body is arranged in a regular shape such as a rectangular column and so on, and the heavy metal compound particles are unevenly distributed in the glass filter body, and the shape of each filter region is shaped by the heavy metal compound particles as shown in FIG. 4.

It will be understood that the above bowtie filter is generally fixedly installed on a base of an accessory of the radiation source, and the radioactive rays emitted from the radiation source generally refer to X-rays or y-rays or the like for scanning lesion portions of the patient. The radioactive rays are invisible light. Therefore, in order to accurately verify the radiation field of the radiation source corresponding to each filter region, it is generally necessary to use a light field lamp to perform an optical path simulation of the radiation source. Since light emitted from the light field lamp is generally visible light, and the bowtie filter in the embodiments of the present disclosure adopts a filter body composed of the light-transmitting substrate and the heavy metal compound particles doped in the light-transmitting substrate, a light-transmitting property of the light-transmitting substrate for visible light may be utilized to perform an optical path simulation of the light field lamp without disassembling the bowtie filter, thereby facilitating a verification operation of the radiation field of the radiation source.

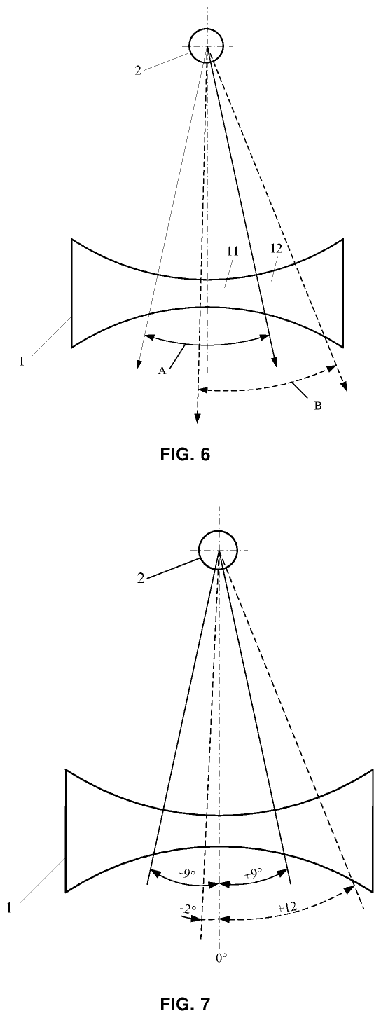

Based on the above bowtie filter, the embodiments of the present disclosure further provide a radiation scanning apparatus. Referring to FIGS. 6 and 7, the radiation scanning apparatus includes a radiation source 2 and the bowtie filter provided by the above embodiment. The bowtie filter is disposed at a light outlet side of the radiation source 2, and each filter region of the bowtie filter corresponds to a different irradiation field of the radiation source 2.

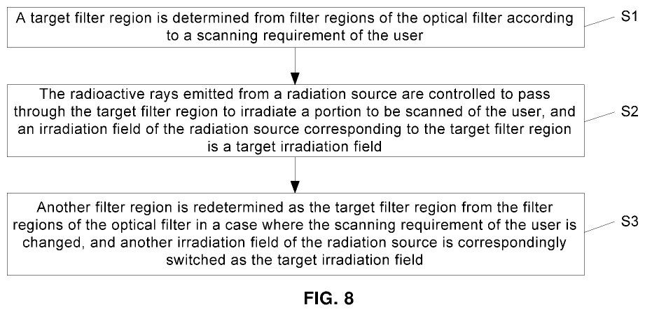

In a case where the radiation scanning apparatus provided in the above embodiment is used, referring to FIG. 8, the radiation scanning method includes the following steps.

In step S1, a target filter region is determined from filter regions of the bowtie filter according to a scanning requirement of the user.

For example, referring to FIG. 6, the filter body 1 of the bowtie filter has two filter regions, one of which is a first filter region 11 used for radiation compensation of the head to be scanned, and another is a second filter region 12 used for radiation compensation of the body to be scanned. If the user's head needs to be scanned, the first filter region 11 is selected as the target filter region. If the user's body needs to be scanned, the second filter region 12 is selected as the target filter region.

In step S2, the radioactive rays emitted from a radiation source are controlled to pass through the target filter region to irradiate a portion to be scanned of the user, and an irradiation field of the radiation source corresponding to the target filter region is a target irradiation field.

With continued reference to FIG. 6, if the target filter region of the above bowtie filter is the first filter region 11, the target irradiation field of the radioactive rays emitted from the radiation source 2 corresponds to a head irradiation field A. And, if the target filter region of the bowtie filter is the second filter region 12, the target irradiation field of the radioactive rays emitted from the radiation source 2 corresponds to a body irradiation field B.

In step S3, another filter region is redetermined as the target filter region from the filter regions of the bowtie filter in a case where the scanning requirement of the user is changed, and another irradiation field of the radiation source is correspondingly switched as the target irradiation field.

With continued reference to FIG. 6, if the target filter region determined in step S1 is the first filter region 11, and the scanning requirement of the user is changed at this time, that is, the user's body needs to be scanned at this time, the second filter region 12 needs to be redetermined as the target filter region. The radioactive rays emitted from the radiation source 2 are controlled to pass through the second filter region 12 to irradiate the user's body to be scanned, and the target irradiation field of the radioactive rays emitted from the radiation source 2 is adjusted from the original head irradiation field A to the body irradiation field B.

It will be noted that, ranges of regions covered by the head irradiation field A and the body irradiation field B respectively may be specifically determined according to the actual needs of the user's head scanning or body scanning. For example, referring to FIGS. 6 and 7, in a case where the filter body 1 in the bowtie filter provided by the embodiments of the present disclosure adopts a bow column structure as shown in FIG. 6, the filter body 1 is symmetrically arranged with a line connecting the light source center of the radiation source 2 and the center of the bow of the filter body 1 as a central axis. The head irradiation field A takes the light source center of the radiation source 2 as a vertex, and is angularly and symmetrically covered on both sides of the central axis. For example, in a case where the central axis is located at 0°, the head irradiation field A is −9° to +9° as shown in FIG. 7. The body irradiation field B may be partially coincident with the head irradiation field A, and the body irradiation field B takes the light source center of the radiation source 2 as a vertex, and is angularly covered on one side or both sides of the central axis. For example, in a case where the central axis is located at 0° , the body irradiation field B is −2° to +12° as shown in FIG. 7. The body irradiation field B takes the light source center of the radiation source 2 as a vertex, and may cover a radiation irradiation generally greater than or equal to 14°.

The beneficial effects that may be achieved by the radiation scanning apparatus and the radiation scanning method provided by the embodiments of the present disclosure are the same as the beneficial effects of the bowtie filter provided by the above embodiments, which will not be described here again.

The foregoing descriptions are merely specific implementation manners of the present disclosure, but the protection scope of the present disclosure is not limited thereto. Any person skilled in the art can readily conceive of changes or replacements within the technical scope of the present disclosure should all be included in the protection scope of the present disclosure. Therefore, the protection scope of the present disclosure should be subject to the protection scope of the claims.

1. A bowtie filter, comprising:
a filter body, wherein includes at least two filter regions; each of the at least two filter regions is in contact with or partially coincident with an adjacent filter region, and every two adjacent filter regions have different radiation compensation amounts.

2. The bowtie filter according to claim 1, wherein the at least two filter regions include at least one first filter region configured for radiation compensation of a head to be scanned, and at least one second filter region configured for radiation compensation of a body to be scanned; and
a surface of the first filter region configured for facing a radiation source is a first curved surface, a surface of the second filter region configured for facing the radiation source is a second curved surface, and a curvature center of the first curved surface and a curvature center of the second curved surface are in a same straight line or coincident.

3. The bowtie filter according to claim 2, wherein curvature of the first curved surface is the same as curvature of the second curved surface, or a curvature variation of the first curved surface is the same as a curvature variation of the second curved surface.

4. The bowtie filter according to claim 2, wherein curvature of the first curved surface is different from curvature of the second curved surface, or a curvature variation of the first curved surface is different from a curvature variation of the second curved surface.

5. The bowtie filter according to claim 2, wherein curvature of a curved surface allowable for the radiation compensation of the head to be scanned is a head applicable curvature ρt₁, and curvature of a curved surface allowable for the radiation compensation of the body to be scanned is a body applicable curvature ρt₂; and
the curvature of the first curved surface and the curvature of the second curved surface are both the same as curvature ρ, wherein ρt₂ is less than or equal to ρ, and ρ is less than ρt₁.

6. The bowtie filter according to claim 2, wherein a number of the first filter regions is one, and a number of the second filter regions is two; and
the two second filter regions are respectively disposed on both sides of a the first filter region.

7. The bowtie filter according to claim 6, wherein curvature of the second curved surfaces of the two second filter regions are the same or different; or
curvature variations of the second curved surfaces of the two second filter regions are the same or different.

8. The bowtie filter according to claim 1, wherein the filter body is a columnar filter body, and a cross section of the columnar filter body is in a bow-tie shape.

9. The bowtie filter according to claim 1, wherein the filter body includes at least one of an aluminum filter body, a ceramic filter body or a teflon filter body.

10. The bowtie filter according to claim 1, wherein the filter body includes a light-transmitting substrate, and the light-transmitting substrate is doped with heavy metal compound particles.

11. The bowtie filter according to claim 10, wherein each filter region is formed by shaping the heavy metal compound particles in a corresponding region in the light-transmitting substrate, and one filter region has one corresponding region.

12. A radiation scanning apparatus, comprising:
a radiation source;
a bowtie filter, wherein the bowtie filter includes a filter body, and the filter body includes at least two filter regions;
each of the at least two filter regions is in contact with or partially coincident with an adjacent filter region, and every two adjacent filter regions have different radiation compensation amounts; and
the bowtie filter is disposed at a light outlet side of the radiation source, and each filter region of the bowtie filter corresponds to a different irradiation field of the radiation source.

13. A radiation scanning method, comprising:
determining a filter region from filter regions of the bowtie filter as a target filter region according to a scanning requirement of a user;
controlling radioactive rays emitted from a radiation source to pass through the target filter region to irradiate a portion to be scanned of the user, an irradiation field of the radiation source corresponding to the target filter region being a target irradiation field; and
redetermining another filter region as the target filter region from the filter regions of the bowtie filter in a case where a scanning requirement of a user is changed, and correspondingly switching another irradiation field of the radiation source as the target irradiation field.

14. The radiation scanning method according to claim 13, wherein in a case where the target filter region of the bowtie filter is a first filter region configured for radiation compensation of a head to be scanned, the target irradiation field of the radiation source is a head irradiation field; and
in a case where the target filter region of the bowtie filter is a second filter region configured for radiation compensation of a body to be scanned, the target irradiation field of the radiation source is a body irradiation field.

15. The bowtie filter according to claim 4, wherein the first curved surface is smoothly in contact with an adjacent second curved surface in a case where the first curved surface is disposed adjacent to the second curved surface.

16. The radiation scanning method according to claim 13, wherein curvature of a first curved surface and curvature of a second curved surface are the same or different; and
a curvature variation of the first curved surface and a curvature variation of the second curved surface are the same or different.

17. The radiation scanning method according to claim 14, wherein curvature of a curved surface allowable for the radiation compensation of the head to be scanned is a head applicable curvature ρt₁, and curvature of a curved surface allowable for the radiation compensation of the body to be scanned is a body applicable curvature ρt₂; and
curvature of a first curved surface and the curvature of a second curved surface are both the same as curvature ρ, wherein ρt₂ is less than or equal to ρ, and ρ is less than ρt₁.

18. The radiation scanning method according to claim 14, wherein a number of the first filter regions is one, and a number of the second filter regions is two; and
the two second filter regions are respectively disposed on both sides of the first filter region.

19. The radiation scanning method according to claim 18, wherein curvature of second curved surfaces of the two second filter regions are the same or different; or
curvature variations of the second curved surfaces of the two second filter regions are the same or different.
